(19) United States
(12) Patent Application Publication
(10) Pub. No.: US 2025/0345013 A1
(43) Pub. Date: Nov. 13, 2025

(54) DEVICE FOR SIGNALLING A ZONE HAVING PREDETERMINED DIMENSIONS AROUND THE DEVICE

(71) Applicant: ASSISTANCE PUBLIQUE - HOPITAUX DE PARIS, Paris 12 (FR)

(72) Inventor: Radouane BARKALLAH, Bagneux (FR)

(21) Appl. No.: 18/862,704

(22) PCT Filed: May 22, 2023
(86) PCT No.: PCT/EP2023/063690

§ 371 Date: Nov. 4, 2024

(30) Foreign Application Priority Data
May 23, 2022 (FR) FR2204944

(51) Int. Cl. A61B 6/10; A61B 6/00

(52) U.S. Cl. CPC A61B 6/107; A61B 6/4405

(57) ABSTRACT

A device for signaling a zone having predetermined dimensions around the device, the device includes emission structure for emitting a light beam towards a projection surface so as to form a light image thereon; measuring structure for measuring the distance between the emission structure for emitting the light beam and the projection surface; and a control unit for determining, on the basis of the measured distance and the predetermined parameters of the zone to be signaled, the arrangement of the emission structure so as to emit a light beam whose light image on the projection surface visibly defines a zone whose dimensions correspond to the zone to be signaled having predetermined dimensions around the device.

The present invention relates to the field of radiology and in particular to the field of personal safety in relation to the rays emitted during radiology operations.

Hospitals have many radiology stations. Radiography is used in particular to visualize disorders associated with the chest and injuries to the musculoskeletal system (bones, joints, etc.). It is involved in the detection and treatment of inflammatory diseases, heart failure and pneumonia. It is also used virtually systematically in orthopedics, where it can be used to highlight skeletal injuries.

The majority of X-rays are taken in restricted areas, where only the patient is present during the examination and other people, such as healthcare professionals, are outside the examination area, thereby ensuring they are protected from the radiation.

However, in hospitals, bedside radiology is also frequently performed, when the patient is unable to move about, for example. This means that radiology staff go to the patient's bedside with mobile radiology equipment. The aim is thus to produce the X-ray required by the medical prescriber (radiologists, surgeons, doctors and anesthetists) on a patient in their room, in the emergency department, or even in the operating room.

While, according to the Institut de Radioprotection et de Sûreté Nucleaire (IRSN) [Institute for Radiation Protection and Nuclear Safety], “bedside X-rays should only be used on patients who cannot be transported, and unnecessary image capture should be avoided”, bedside radiography alone represents an enormous sector of activity, as can be seen in every hospital. For example, a technician working in a Parisian hospital such as Lariboisière can perform an average of forty “bedside” X-rays each morning.

As a result, these “mobile” X-rays can be taken anywhere, whether in a ward room or in recovery after surgery, and, according to IRSN, when taking the image, “the operator must stand at the maximum distance compatible with proper performance of the procedure (cord length, remote control, room dimensions and layout), and wear an X-ray protection apron”. This protection zone measures on average one and a half meters around the X-ray emitting tube.

Thus, although these procedures are carried out in premises that are not classified as a “restricted zone”, regulations require temporarily defining a controlled zone, known as a safety or operating zone, around the mobile apparatus.

Radiation protection and the safety zone are therefore fields that radiology technicians must master, and they have been trained to do so. However, this is not the case for other healthcare professionals (nurses, auxiliary nurses) or for the families who may be present during the bedside X-ray. As a result, the only information healthcare professionals and/or other people have regarding radiation protection is that transmitted by the people carrying out the bedside X-ray.

In document EP-A-3297538, a device is proposed for signaling the status of an outdoor radio-frequency emitting apparatus, in particular equipped with an X-ray tube. This apparatus is connected to the power supply cable of the radiology device and includes current measurement means for defining the status of said apparatus (in use, off, on standby). It also includes means for transmitting the measurement to a second part for signaling the status of the apparatus. Signaling devices may be audible (alarm), light-based (indicator light) or display messages. However, although such a device can be used to warn whether or not an apparatus equipped therewith is operating, it does not make it possible to estimate the safety zone around the apparatus.

As already indicated, when radiography is carried out outside a regulated examination room, the zoning is given solely by the person in charge of the procedure, and the technician has to remove all people from the X-ray perimeter before performing the radiography on the patient, on the basis of an estimate of the necessary distance.

The first difficulty is therefore that the radiology technician has only their say-so to create distance and warn people present in the room where the x-ray is to be taken.

In addition, it is sometimes difficult to estimate the actual dimensions of the safety or protection zone around the apparatus in a space which is not designed for this purpose and which may be cluttered.

Document KR 2016 0004621 describes a device for indicating a safety zone around a radiation-emitting device such as a mobile X-ray apparatus, comprising a laser emitter associated with an angular adapter to form a laser guide line on the ground that is visible to a user. The laser unit comprises a light-receiving sensor, a multicolor diode and a control unit so that, when the sensor detects an object, the laser unit notifies an approaching object by emitting different colors. In addition, a radiation measurement unit detects the amount of radiation emitted, and this measurement is used to modify the angle of inclination of the laser unit to modify the safety zone around the radiation-emitting apparatus. The safety zone indicating device is in the form of a frame which is installed around the apparatus in the form of a rectangular radiation-emitting block. Thus, the frame can be adjusted around the apparatus which is the source of radiation, and features four laser units on each side face of the block, defining a line on each side, with the lines together defining the safety zone thus delimited on the ground. The laser image is thus formed of lines and their positioning is controlled by the inclination of the laser beam emission means, this inclination being controlled and adjustable on the basis of the measured radiation intensity.

EP 2 117 649 proposes a signaling system comprising a control unit for providing control signals in response to a predicted and/or measured spatial distribution of scattered X-rays in an environment of an object in a treatment room; and a signaling unit for providing at least one perceptible signal in response to said control signals, said at least one perceptible signal being indicative of said spatial distribution of scattered radiation.

Thus, the scattered radiation is generated by the object being irradiated with an X-ray beam, this scattered radiation being emitted from the object to its environment outside the beam. The signaling system comprises a control unit for control signals in response to a predicted and/or measured spatial distribution of scattered X-rays in an environment of an object in a treatment room, a signaling unit which provides at least one signal in response to the control signals, said signal being indicative of the spatial distribution of the radiation scattered from the object.

US 2021/0137483 describes systems and methods for monitoring the progress of a medical procedure by exposure to scattered radiation. The locations of the elements of the medical equipment producing and emitting “scattered” radiation are received by a monitoring device. The location of people participating in the medical procedure is received by the monitoring device by means of tracking devices worn by the people during the medical procedure. The monitoring device receives an indication of when the element(s) producing the radiation are activated, and creates a radiation scattering intensity field. An analytical subsystem determines an estimated degree of exposure for each individual based on their position in the scattering intensity field of the scattered radiation each time the radiation-producing device is activated, which is compared with a reference that may be specific to the procedure. When the estimated degree of exposure exceeds the reference value, an intervention subsystem intervenes.

These systems involve relatively complex systems.

The aim of the invention is therefore to provide a device which enables the technician of the “mobile” radiology apparatus to determine the safety zone around the apparatus they are intending to use in a simple and reliable way, in order to protect other people located in the vicinity of the machine by sending them out of this zone.

To this end, the invention relates to a device for signaling a zone having predetermined dimensions around said device, which device is characterized in that it comprises:
emission means for emitting a light beam toward a projection surface so as to form a light image thereon,
means for measuring the distance between the emission means for emitting the light beam and the projection surface,
a control unit for determining, on the basis of the measured distance and predetermined parameters of the zone to be signaled, the arrangement of the emission means so as to emit a light beam whose light image on the projection surface visibly defines a zone whose dimensions correspond to the zone to be signaled having predetermined dimensions around the device.

Thus, advantageously, the signaling device makes a zone having predetermined dimensions around the signaling device (which is substantially at the center thereof) visible on a surface referred to as the projection surface, so that people can see it and thus easily position themselves in relation to this zone; either to leave said zone, in the case of a safety zone which it is necessary to be outside in order to be protected, or to enter said zone in the case of a safety zone which it is necessary to be inside in order to be protected. In the latter case, zones can be provided that delimit a location in which people can be safely positioned, for example under camera surveillance, on a subway platform.

Very advantageously, when the signaling device is used in a room such as a bedroom, a ward or even an operating room, the surface onto which a light image is projected is the ceiling of this room, which is always perfectly visible to people present despite the clutter that may be present in this room (furniture, partitioning curtains, etc.).

In addition, the signaling device according to the invention can be used advantageously in rooms of different dimensions, since the device makes it possible to define an image on the projection surface which corresponds to predetermined parameters regardless of the distance at which the device according to the invention is in relation to this surface.

According to a preferred embodiment, the signaling device is in the form of a housing in which the emission means for emitting the light beam and the means for measuring the distance are housed adjacently on the same face of said housing and are positionable opposite the projection surface. Advantageously, the distance between the measuring means and the projection surface is identical to the distance between the emission means for emitting the light beam and said projection surface.

In one embodiment, the signaling device is associated with a radiology apparatus, preferably a mobile radiology apparatus, for which an operating or safety zone has to be defined when it is in use.

Preferably, therefore, the parameters of the zone having predetermined dimensions that the device according to the invention makes it possible to signal by visualization are the parameters of a safety zone around a radiology apparatus, the signaling device according to the invention being attached to the X-ray tube, for example. These parameters are entered and stored in the control unit.

The signaling device according to the invention, associated with a mobile radiology apparatus, makes it possible to make the safety zone around the radiology apparatus visible, regardless of the room in which they are located and the height of the ceiling on which the light image is formed, the safety zone which is made visible always being the same, irrespective of the room.

Thus, on the orders of the radiology apparatus technician, people present in the room can easily move outside the safety zone visible on the ceiling, in order to place themselves out of range of the rays from the radiology apparatus.

It is thus possible for healthcare professionals to also move other

patients away by pushing beds or gurneys out of the visible safety perimeter defined on the ceiling.

The invention also relates to a radiology apparatus, preferably a mobile radiology apparatus, which has a signaling device according to the invention attached thereto such that, when the radiology apparatus is in use in a room, the measuring means and the means for projecting a light beam are directed toward the same projection surface, such as the ceiling of the room.

Preferably, the housing of the device according to the invention is provided with means for releasable attachment to the radiology apparatus, preferably magnetic attachment means. Thus, advantageously, the device according to the invention makes it possible to equip existing mobile radiology apparatuses, giving them a degree of operational safety that they did not have beforehand.

Preferably, the device comprises an attachment support, comprising a base provided with two protruding arms, the housing being pivotably mounted at the end of said arms. The housing can thus be oriented by pivoting so that the face provided with the emission means for emitting the light beam is opposite the projection surface.

Even more preferably, the device comprises, on the face of the housing opposite the face comprising the light source, a level for orienting the face of the housing comprising the light source in order for it to be parallel to the projection surface. The face of the housing provided with the emission means and measurement means can thus be positioned so as to be parallel to the ceiling, for example.

The device is attached magnetically to the radiology apparatus, which comprises a magnetized base to which the device is attached.

More advantageously, the device according to the invention can equip any type of mobile apparatus that requires a safety zone around its location.

It can also be envisaged that said device according to the invention is integrated into a radiology apparatus. To this end, according to a second aspect of the invention, it is also a subject of the invention to propose a mobile radiology apparatus, characterized in that it comprises a signaling device as described above, said signaling device being integrated into the radiology apparatus such that, when the latter is in use in a room, the measuring means and the means for projecting a light beam can be directed toward the same projection surface, such as the ceiling of the room.

Advantageously, such a radiology apparatus is equipped with the device for signaling what is referred to as the safety zone for its use, regardless of the location where it is used.

According to one embodiment of the invention, the measuring means use optical, acoustic or radio-frequency methods to measure the distance between them and a distant object. The means for measuring the distance thus consist of optical, acoustic or radio-frequency means for measuring the distance between the signaling device and the projection surface.

According to a preferred embodiment, the means for measuring the distance consist of optical means for measuring the distance between the signaling device and the projection surface, said measuring means preferably being adjacent to the emission means for emitting the light beam, said optical measuring beam and said light beam extending in parallel.

Preferably, these distance-measuring means consist of a telemeter projecting an optical beam, such as a laser or infrared beam, making it possible to measure the distance from the device to a surface, such as the ceiling of the room in which the apparatus is located.

The emission means for emitting a light beam consist of a laser source and an optical system for emitting a laser beam, preferably a divergent laser beam, which forms, on the surface, a light image in the form of a preferably colored light circle, for example red, green or blue depending on the visible laser source, having a radius of 2 m corresponding to a desired safety zone.

The measuring means, such as the telemeter, and the emission means for emitting the light beam are in communication, the telemeter sending distance data to the control unit which controls the emission means, the focal distance and laser source intensity of which can thus be adjusted so that the light circle represented on the ceiling always has a radius of two meters regardless of the distance measured between the ceiling and the device. Preferably, the device according to the invention forms a light circle having a radius of 2 meters, with a distance between the device and the ceiling of approximately 35 cm, for example.

The choice of a radius of two meters includes a safety margin in relation to regulations (1.50 m), in order to reinforce the protection.

The emission means for emitting a light beam can consist of a light source consisting of light-emitting diodes (LEDs) and an optical system, the light beam emitted in this way forming, on the projection surface, a light image in the form of a preferably colored circular light spot, the focal length and light intensity of the light beam of said emission means being adjustable by the control unit.

The invention will now be described in greater detail with reference to the figures, which depict:

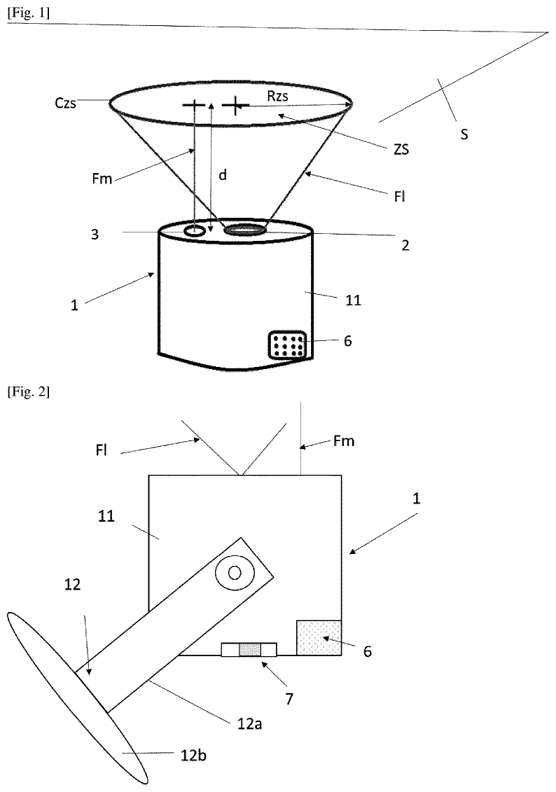

FIG. 1 A schematic perspective view of the top of the housing of a device according to the invention;

FIG. 2 A schematic side view of the device of the invention;

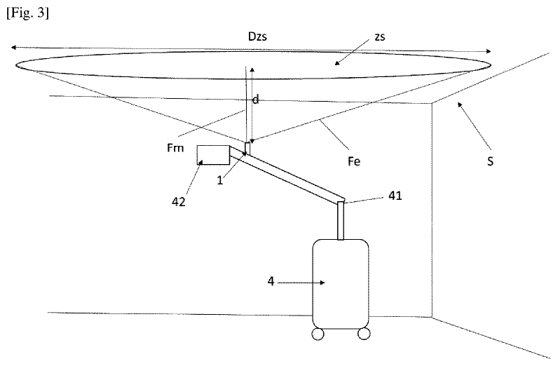

FIG. 3 A schematic perspective view of a room in which a mobile radiology apparatus equipped with a device according to the invention is located.

The signaling device 1 according to the invention is in the form of a housing 11 in which the emission means 2 for emitting a light beam Fl toward a projection surface S and the means 3 for measuring the distance d are provided between said device 1 and the surface S.

In a preferred embodiment of the invention, the measuring means 3 consist of a telemeter, preferably an optical telemeter. This telemeter is a laser telemeter that projects a laser beam as a measuring beam Fm toward the surface S, which in turn reflects the light beam.

The electronic processing means of the telemeter calculate the phase shift between emission and reception of the measuring beam Fm and thus determine the distance d. Use may also be made of an infrared telemeter or an acoustic (ultrasound) telemeter.

The emission means 2 for emitting the light beam Fl and the means 3 for measuring the distance d are housed in the housing 11, on the same face of said housing 11 which can be positioned opposite the surface S, such that the distance d between the measuring means 3 and the surface S is identical to the distance d between the emission means 2 and said surface S.

The measuring beam Fm is thus emitted toward the surface S, with the telemeter then measuring the distance d. The value of the distance d is sent to the emission means 2 for emitting the light beam, which include a unit for controlling the value of this distance d. This control unit determines, on the basis of the distance d and also of the parameters of the safety zone ZS to be signaled, the appropriate arrangement of said emission means 2. In this way, said emission means then emit a light beam Fl forming a light image on the surface S which defines a zone whose dimensions correspond to the predetermined parameters for a safety zone ZS around said device 1.

The device further comprises an attachment support 12, comprising a base 12b provided with two protruding arms 12a, the housing 11 being pivotably mounted at the end of said arms. A base, for example a magnetized base, is attached, for example adhesively bonded, to the mobile radiology apparatus, at the X-ray tube 42 or the support arm 41 thereof. The attachment support 12 is then magnetically attached to the base.

The device 1 also includes, on its face opposite the face comprising the light source, a level 7 making it possible to adjust the positioning of the housing 11 relative to the arms 12a, and thus its orientation, so that the face of the housing 11 comprising the light source is parallel to the ceiling.

The emission means 2 for emitting the light beam FI consist of a divergent laser beam forming a beam which, projected onto the surface S, forms a light image in the form of a preferably colored light circle Czs. The control unit makes it possible, on the basis of the distance d and the predetermined parameters of the safety zone ZS to be visualized which are stored in said control unit, to adjust the focal length of the laser source and the intensity of the laser in order to form the appropriate light image, in this case the circle Czs.

This circle Czs visibly delimits, on the ceiling (surface S), the circular safety zone ZS around the signaling device 1, this zone being of identical dimensions regardless of the value of the distance d, the unit for controlling the distance d making it possible to adjust the emission means 2 in this regard.

Preferably, when the signaling device 1 is associated with a mobile

radiology apparatus 4, this safety zone ZS is a circular surface delimited by the light circle Czs having the radius Rzs of 2 m, with the device 1 being located at the center of this zone.

Thus, as shown in FIG. 3, a mobile radiology apparatus 4 is brought into a room such as a bedroom to perform a bedside X-ray on a patient. This device 4 comprises an articulated arm 41 at the end of which an X-ray tube 42 is mounted and to which the signaling device 1 according to the invention is attached, with its measuring means 3 and its emission means 2 for emitting a light beam Fl facing the projection surface S, here consisting of the ceiling of the bedroom. The signaling device 1 is as close as possible to the X-ray tube 42 in order to define the safety zone ZS around it.

People present in the room, such as healthcare professionals, can therefore clearly see the light image forming the circle Czs which delimits the safety zone around the X-ray tube 41, enabling them to position themselves outside this circle Czs.

In order to warn those present that an X-ray image will be taken, and to instruct them to leave the safety zone ZS, the signaling device 1 comprises audible alarm means 6 which can be activated by the X-ray technician using a remote actuation button, for example, which can be adhesively bonded to a control unit such as a switch that activates the rays and making it possible to emit an audio signal and give instructions to the patient when the X-rays are triggered.

A remote control may also be provided for remote actuation of the device according to the invention.

This audible alarm actuated by the technician makes it possible, in addition to signal the safety zone, to trigger an audio signal before the X-rays are triggered, so that people can evacuate the safety zone ZS, then, when triggered again when the X-ray is taken, it enables the patient to follow the instructions given to them by the X-ray technician. Indeed, this audible signal emitted when an image is captured (at the moment of “shooting”) can help the patient to better perceive the moment when they need to hold their breath. This could help reduce the number of non-contributory images, and thereby reduce the number of exposures to X-rays.

Thus, when a patient is admitted to the recovery room, the head anesthetist may decide to call the radiology technician to perform a chest X-ray at this patient's bedside, to check the condition of the patient's lungs. The radiology technician goes to the recovery room. The recovery room may be full, and patients are simply separated by screens.

In addition, a large number of healthcare professionals are located in the recovery room. The X-ray technician goes to the bedside of the patient, who is located between two other patients, for example, one of whom is receiving a perfusion from a nurse. The X-ray technician arrives with their equipment (X-ray cassette, X-ray emitting equipment and the device according to the invention attached to the arm 41 as in FIG. 3) and gets set up.

When actuating the device according to the invention, the technician checks, using the level 7, that the housing 11 is parallel to the ceiling of the recovery room and, if necessary, positions the housing 11 correctly.

At the housing 11, the telemeter 3 sends distance d data to the emission means 2 for emitting the laser beam, which adapt the focal length and intensity of the laser so that the zone shown on the ceiling always has a radius Rzs of two meters. Once the safety zone ZS has been projected onto the ceiling S, the audible alarm 6 is actuated by the radiology technician.

Thus, in this situation, the safety zone ZS for X-rays is visible and audible, making it possible to optimize radiation protection for patients in the vicinity, with these patients being informed in this way whether or not they are in the safety zone. If they are inside the safety zone, it will merely be necessary to move their bed. Furthermore, the healthcare professionals in the area will not have to stop their caregiving if they are outside this zone. Finally, the triggering of the X-rays will be heard by everyone present by virtue of the audible alarm.

The signaling device according to the invention therefore advantageously makes it possible to limit the exposure of neighboring patients to X-rays during bedside X-rays, by enabling healthcare professionals to move them outside of the safety zone if necessary.

It is thus possible to reduce staff exposure to X-rays during bedside radiography.

Such a device also makes it possible to optimize the organization of care when taking X-rays and to improve relations among healthcare professionals during radiography at the patient's bedside.

These actions advantageously make it possible to reduce the stress associated with taking bedside X-rays and the risks of excessive exposure to X-rays. Patients also benefit from a better understanding of the instructions given when taking X-ray images.

Furthermore, it is thus possible to contribute to better managing potential disruptions associated with bedside X-rays, such as moving neighboring beds to several meters away from the radiology apparatus and potential conflicts with families present at the time of the examinations.

1. A device for signaling a zone having predetermined dimensions around said device said device comprising:
emission means for emitting a light beam toward a projection surface so as to form a light image thereon;
means for measuring the distance between the emission means for emitting the light beam and the projection surface; and
a control unit for determining, on the basis of the measured distance and predetermined parameters of the zone to be signaled, the arrangement of the emission means so as to emit a light beam whose light image on the projection surface visibly defines a zone whose dimensions correspond to the zone to be signaled having predetermined dimensions around the device.

2. The signaling device according to claim 1, characterized in that the means for measuring the distance consist of optical, acoustic or radio-frequency means for measuring the distance between the signaling device and the projection surface.

3. The signaling device according to claim 2, characterized in that the means for measuring the distance consist of optical measuring means projecting an optical beam such as a laser or infrared beam, making it possible to measure the distance and extending parallel to the light beam emitted by the emission means.

4. The signaling device according to claim 1, characterized in that the emission means for emitting a light beam consist of a laser source and an optical system for emitting a laser beam, preferably a divergent laser beam, which forms, on the projection surface, a light image in the form of a preferably colored light circle, the focal length and light intensity of the light beam of said emission means being adjustable by the control unit.

5. The signaling device according to claim 1, characterized in that the emission means for emitting a light beam consist of a light source consisting of light-emitting diodes (LEDs) and an optical system, the light beam emitted in this way forming, on the projection surface, a light image in the form of a circular light spot, the focal length and light intensity of the light beam of said emission means being adjustable by the control unit.

6. The signaling device according to claim 1, characterized in that it comprises audible alarm means.

7. The signaling device according to claim 1, characterized in that it is in the form of a housing, the emission means for emitting the light beam and the means for measuring the distance being housed adjacently on the same face of said housing and being positionable facing the projection surface.

8. The signaling device according to claim 7, characterized in that it comprises means for attachment to a radiology apparatus, preferably a mobile radiology apparatus, for which a safety zone must be defined when in use, said safety zone constituting the zone having predetermined dimensions.

9. The signaling device according to claim 8, characterized in that the housing is provided with means for releasable attachment to the radiology apparatus.

10. The signaling device according to claim 7, characterized in that it comprises an attachment support comprising a base provided with two protruding arms, the housing being pivotably mounted at the end of said arms.

11. The signaling device according to claim 7, characterized in that it comprises, on the face of the housing opposite the face comprising the light source, a level for orienting the face of the housing comprising the light source.

12. A radiology apparatus, in particular a mobile radiology apparatus, characterized in that it comprises, attached thereto, a signaling device according to claim 1.

13. A radiology apparatus, in particular a mobile radiology apparatus, characterized in that it comprises a signaling device according to claim 1, said signaling device being integrated into the radiology apparatus such that, when the latter is in use in a room, the measuring means and the emission means for emitting a light beam can be directed toward the same projection surface, such as the ceiling of the room.
